(19) United States
(12) Patent Application Publication
(10) Pub. No.: US 2024/0054327 A1
(43) Pub. Date: Feb. 15, 2024

(54) INFORMATION PROCESSING SYSTEM

(71) Applicant: CANON KABUSHIKI KAISHA, Tokyo (JP)

(72) Inventors: KOHTARO UMEZAWA, Tokyo (JP); RYUTA UEDA, Tokyo (JP); RITSUYA TOMITA, Kanagawa (JP)

(21) Appl. No.: 18/447,886

(22) Filed: Aug. 10, 2023

(30) Foreign Application Priority Data
Aug. 12, 2022 (JP) 2022-128577

(51) Int. Cl. G06N 3/0464; G06N 3/08

(52) U.S. Cl. CPC G06N 3/0464; G06N 3/08; G16H 50/20

(57) ABSTRACT

An information processing system includes a second information processing apparatus configured to communicate with a first information processing apparatus via a network, an inference target data acquisition unit configured to acquire inference target data, and an inference unit configured to perform predetermined inference processing on the inference target data by using a first partial model and a second partial model. The first information processing apparatus includes the first partial model configured to include a first input layer, a first group of intermediate layers including at least one of the intermediate layers in the group of intermediate layers, and a first output layer, and the second information processing apparatus includes the second partial model configured to include a second group of intermediate layers including an intermediate layer different from the intermediate layers included in the first group of intermediate layers in the group of intermediate layers, and the second output layer.

### BACKGROUND OF THE DISCLOSURE

### Field of the Disclosure

The present disclosure relates to an information processing system that performs inference processing or training processing using an inference model.

### Description of the Related Art

There is known a system that applies a machine learning technique to medical images acquired by medical imaging apparatuses (modalities) and medical data such as medical information acquired from medical information systems to make inferences about predetermined diseases (e.g., disease detection, benign/malignant discrimination, prognosis prediction, and risk prediction).

Zihao Liu et al., “Orchestrating Medical Image Compression and Remote Segmentation Networks”, International Conference on Medical Image Computing and Computer-Assisted Intervention—MICCAI 2020, discloses a technique in which, when an image is input, image compression is performed by a first information processing apparatus located locally, and the compression result is transferred to a second information processing apparatus located in a remote cloud service to segment diseases.

Behzad Bozorgtabar et al., “SALAD: Self-Supervised Aggregation Learning for Anomaly Detection on X-Rays”, International Conference on Medical Image Computing and Computer-Assisted Intervention—MICCAI2020, discloses a technique of training a model including an encoder and a decoder using a medical image as an input, and extracting a feature using an inference model (encoder unit).

In the technique described in Zihao Liu et al., when a medical image is segmented, the medical image, which is data to be inferred, is transmitted to a cloud service. In this case, since medical data, such as a medical image, is transmitted from a hospital to an external information processing apparatus, it is difficult to protect privacy of the medical data.

On the other hand, as in the technique described in Behzad Bozorgtabar et al., an inference model may be built in an information processing apparatus managed by a user who performs inference. In such a case, it is difficult to prevent unauthorized use, such as the user copying the inference model and distributing it to a third party, or altering the inference model, and thus confidentiality of the inference model cannot be secured.

### SUMMARY OF THE DISCLOSURE

The present disclosure is directed to providing an information processing system capable of securing confidentiality of an inference model while protecting privacy of medical data.

According to an aspect of the present disclosure, an information processing system includes a first information processing apparatus, a second information processing apparatus configured to communicate with the first information processing apparatus via a network, an inference target data acquisition unit configured to acquire inference target data, and an inference unit configured to perform predetermined inference processing on the inference target data by using a first partial model and a second partial model, wherein the information processing system performs predetermined inference processing by using an inference model based on a neural network including a first input layer, a group of intermediate layers, a first output layer, and a second output layer, the first output layer and the second output layer being provided in different information processing apparatuses, wherein the first information processing apparatus includes the first partial model configured to include the first input layer, a first group of intermediate layers including at least one of the intermediate layers in the group of intermediate layers, and the first output layer, and wherein the second information processing apparatus includes the second partial model configured to include a second group of intermediate layers including an intermediate layer different from the intermediate layers included in the first group of intermediate layers in the group of intermediate layers, and the second output layer.

Further features of the present disclosure will become apparent from the following description of exemplary embodiments with reference to the attached drawings.

### BRIEF DESCRIPTION OF THE DRAWINGS

FIG. 1 is a diagram illustrating a configuration of an information processing system according to a first exemplary embodiment and a third exemplary embodiment.

FIG. 2 is a diagram illustrating a hardware configuration of a first information processing apparatus according to the first exemplary embodiment.

FIG. 3 is a schematic diagram of an inference model according to the first exemplary embodiment.

FIG. 4 is a flowchart illustrating inference processing performed by the information processing system according to the first exemplary embodiment.

FIG. 5 is a diagram illustrating a configuration of an information processing system according to a second exemplary embodiment and a fourth exemplary embodiment.

FIG. 6 is a schematic diagram of an inference model according to the second exemplary embodiment.

FIG. 7 is a flowchart illustrating training processing of an inference model according to the second exemplary embodiment.

FIG. 8 is a configuration diagram of an inference model according to the third exemplary embodiment and the fourth exemplary embodiment.

FIG. 9 is a flowchart illustrating inference processing performed by the information processing system according to the third exemplary embodiment.

FIG. 10 is a flowchart illustrating training processing performed by the information processing system according to the fourth exemplary embodiment.

### DESCRIPTION OF THE EMBODIMENTS

The present disclosure can be desirably applied to raw data (signal data) acquired by a modality, and medical data generated by image reconstruction from raw data, for example, data for diagnosis.

Examples of the modality include an X-ray computed tomographic (CT) scanner, a magnetic resonance imaging (MRI) scanner, a single photon emission computed tomography (SPECT) scanner, a positron emission tomography (PET) scanner, and an electrocardiograph. Inference target data and training data may include not only medical data but also information related to privacy of a patient, such as age, sex, and disease information. Further, the inference target data is not limited to the medical data. The inference target data may be any data, such as image data representing a person, text data based on document data, and voice data based on voice, as long as inference processing can be performed over a neural network.

An inference process using an inference model in the information processing system of the present disclosure will be described below in first and third exemplary embodiments. The first exemplary embodiment and the third exemplary embodiment are different from each other in an inference model for performing inference and a procedure of inference processing. In second and fourth exemplary embodiments, a training process of an inference model in the information processing system of the present disclosure will be described. As will be described below, the inference model used in the inference process is not limited to the inference model generated through the training process according to at least one of the second and fourth exemplary embodiments of the present disclosure. The inference model used in the inference process is a trained inference model trained based on machine learning or deep learning by a known method or the training process described in the present disclosure. Here, the trained inference model may be used as a target of additional learning, transfer learning, or fine tuning as long as training processing is performed to satisfy a predetermined condition. Therefore, as additional learning of a trained inference model trained by a known method, training processing by a training process to be described below may be performed, or the training processing may be performed in reverse order.

Hereinafter, exemplary embodiments of the present disclosure will be described with reference to the accompanying drawings.

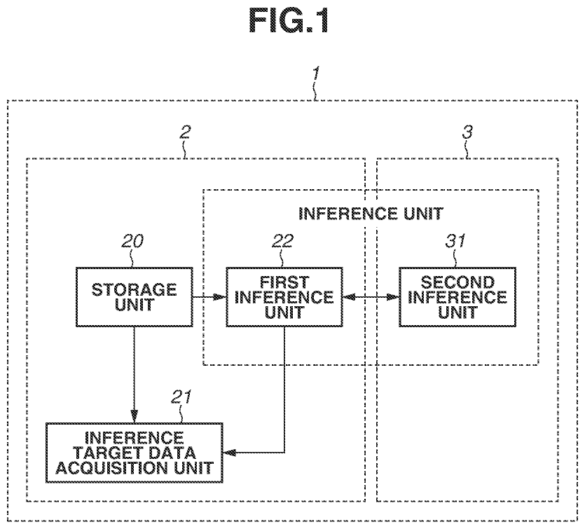

An information processing system 1 according to the first exemplary embodiment of the present disclosure will be described with reference to FIG. 1. The information processing system 1 according to the present exemplary embodiment of the present disclosure includes a first information processing apparatus 2 and a second information processing apparatus 3 capable of communicating with the first information processing apparatus 2 via a network.

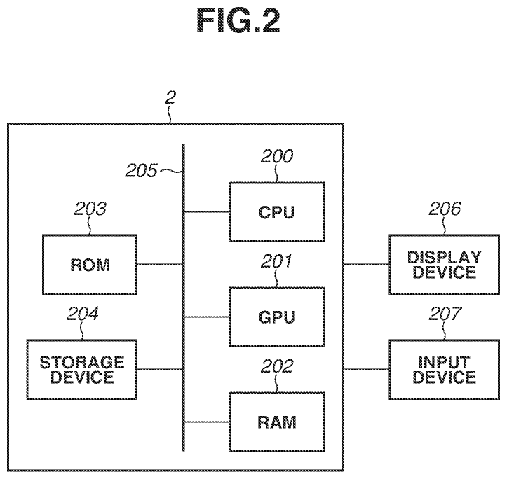

FIG. 2 illustrates an example of a specific configuration of the first information processing apparatus 2. In the example, the first information processing apparatus 2 includes a central processing unit (CPU) 200, a graphics processing unit (GPU) 201, a random access memory (RAM) 202, a read only memory (ROM) 203, and a storage device 204, which are connected by a system bus 205. A display device 206 and an input device 207, such as a mouse and a keyboard, are connected to the first information processing apparatus 2. The second information processing apparatus 3 may be configured in the same manner as the first information processing apparatus 2, or may be configured to include a part of the configuration of the first information processing apparatus 2.

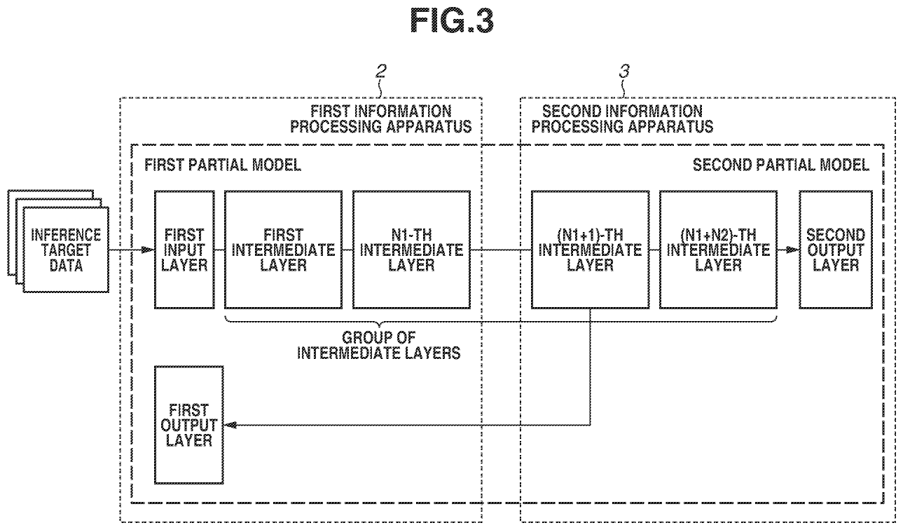

A functional configuration of the information processing system 1 according to the present exemplary embodiment of the present disclosure and the inference model will be described below with reference to FIG. 3. The information processing system 1 includes the first information processing apparatus 2 and the second information processing apparatus 3 capable of communicating with the first information processing apparatus 2 via a network. Here, for example, the first information processing apparatus 2 is an information processing apparatus managed by a user of the inference model, and the second information processing apparatus 3 is an information processing apparatus managed by a provider of the inference model. The first information processing apparatus 2 and the second information processing apparatus 3 each include a partial model that is a part of an inference model that performs inference processing on inference target medical data and outputs an execution result. The inference model is a neural network configured to include a first input layer, a group of intermediate layers, a first output layer, and a second output layer, and the first output layer is provided as a configuration of the partial model of the first information processing apparatus 2. The second output layer is provided as a configuration of the partial model of the second information processing apparatus 3. In a process of the inference processing, the inference model is a trained inference model trained through predetermined training processing. A parameter for outputting an inference result is determined by the training processing, and a model in which the parameter and a network model are paired is defined as the inference model.

The first information processing apparatus 2 includes a first partial model configured to include a first input layer, a first group of intermediate layers which includes at least one of the intermediate layers in the group of intermediate layers, and a first output layer in the above-described inference model. For example, as the first group of intermediate layers, as illustrated in FIG. 3, N1 intermediate layers from the first intermediate layer to an N1-th intermediate layer are provided.

The second information processing apparatus 3 includes a second partial model configured to include a second group of intermediate layers including intermediate layers different from the intermediate layers constituting the first group of intermediate layers in the group of intermediate layers, and a second output layer. For example, as the second group of intermediate layers, as illustrated in FIG. 3, N2 intermediate layers from an (N1+1)-th intermediate layer to an (N1+N2)-th intermediate layer are provided. Here, the first information processing apparatus 2 and the second information processing apparatus 3 may have respective intermediate layers each having a common parameter. For example, in addition to the above, the first group of intermediate layers and the second group of intermediate layers may have N3 common intermediate layers from an (N1+N2+1)-th intermediate layer to an (N1+N2+N3)-th intermediate layer, which are not illustrated in FIG. 3. Hereinafter, the configuration of each of the information processing apparatuses will be described.

The first information processing apparatus 2 is an information processing apparatus that can be operated by, for example, a medical service worker who is a user of an inference model and has authority to manage inference target medical data. On the other hand, the second information processing apparatus 3 is an information processing apparatus owned by the provider of the inference model who has authority to manage an inference model used for inference. The second information processing apparatus 3 exists in a server outside the first information processing apparatus 2 and is configured to be able to communicate via a network.

The first information processing apparatus 2 includes a storage unit 20 for storing model information about the first partial model and inference target data. The storage unit 20 may be configured to be an external device of the first information processing apparatus 2. The first information processing apparatus 2 includes an inference target data acquisition unit 21 that acquires inference target medical data, and a first inference unit 22 that performs first inference processing using the first partial model, among inference units that perform inference processing on the inference target medical data.

The second information processing apparatus 3 includes a second inference unit 31 that exists in a server outside the first information processing apparatus 2, receives the result of the first inference processing from the first information processing apparatus 2, and executes second inference processing by the second partial model, among the inference units that perform inference processing.

The information processing system 1 according to the present exemplary embodiment of the present disclosure performs the inference processing in which the inference model based on the neural network including the first input layer, the group of intermediate layers, the first output layer, and the second output layer is divided into the first partial model of the first information processing apparatus 2 and the second partial model of the second information processing apparatus 3, so that the confidentiality of the inference model can be secured while the privacy of the medical data is protected. Specifically, the user of the inference model who is an administrator of the inference target data does not need to transmit the inference target medical data to an external information processing apparatus, and can obtain an inference result while protecting the privacy of the medical data.

The provider of the inference model can secure the confidentiality of the inference model by installing only a part of the inference model in the first information processing apparatus 2.

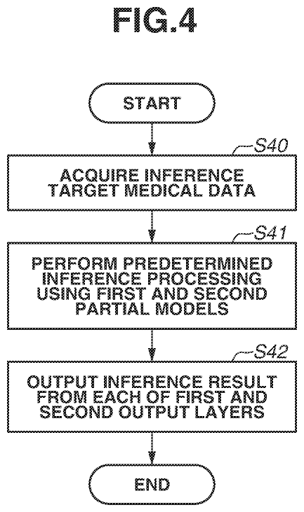

An inference process performed by the information processing system 1 according to the present exemplary embodiment will be described with reference to FIG. 4.

In step S40, the inference target data acquisition unit 21 in the first information processing apparatus 2 acquires inference target medical data, transmits the acquired inference target medical data to the first inference unit 22 constituting the inference unit, and the processing proceeds to the next step.

In step S41, the inference unit performs predetermined inference processing using the first partial model and the second partial model. Specifically, the inference target medical data is input to the first input layer constituting the first inference unit 22, the inference processing of the first intermediate layer to the N1-th intermediate layer is executed, and intermediate information is transmitted to the second inference unit 31. The second inference unit 31 receives the intermediate information transmitted from the first inference unit 22, and performs inference processing by the (N1+1)-th intermediate layer to the (N1+N2)-th intermediate layer. Then, when an inference result by the (N1+N2)-th intermediate layer is transmitted to the second output layer constituting the second partial model and the first output layer constituting the first partial model, the processing proceeds to the next step.

In the present embodiment, the (N1+N2)-th intermediate layer is an example of a final intermediate layer, which is the final layer constituting the group of intermediate layers. However, the (N1+N2)-th intermediate layer may not be the final intermediate layer, and an (N1+N2+1)-th intermediate layer to a final intermediate layer may be common intermediate layers in each of the partial models. At this time, the inference result by the (N1+N2)-th intermediate layer is transmitted to the (N1+N2+1)-th intermediate layer constituting each of the partial models. Then, the processing from the (N1+N2+1)-th intermediate layer to the final intermediate layer is executed, and an inference result by the final intermediate layer is transmitted to the respective output layers.

In step S42, the first output layer of the first partial model and the second output layer of the second partial model each receive the intermediate information from the (N+2)-th intermediate layer as an input, and output an inference result for predetermined inference processing. Since the inference result is also output to the second information processing apparatus 3, which is managed by a distributor of the inference model, it is possible to prevent unintended inference processing. Specifically, when an image unintended by the provider who is a distribution source of the inference model is input by the user who is a distribution destination of the inference model, for example, when inference processing is performed a plurality of times by adding a little noise information, the inference processing can be detected from a likelihood distribution of outputs by the second output layer or a difference between results of the inference processing performed the plurality of times by the second output layer. When detecting the unintended inference processing, the second information processing apparatus 3 stops the use of the inference model. Specifically, input of information to the intermediate layer provided only in the second information processing apparatus 3 is not permitted. With this configuration, it is possible to secure the confidentiality of information about the intermediate layer constituting the second partial model of the second information processing apparatus 3.

The information processing system 1 according to the present exemplary embodiment of the present disclosure can protect the privacy of the inference target medical data and secure the confidentiality of the inference model for performing inference on the inference target medical data by implementing the above-described inference model and an inference processing process.

In the first exemplary embodiment, the inference processing using the inference model based on the neural network including the first input layer, the group of intermediate layers, the first output layer, and the second output layer has been described. In the second exemplary embodiment, training processing using the inference model will be described.

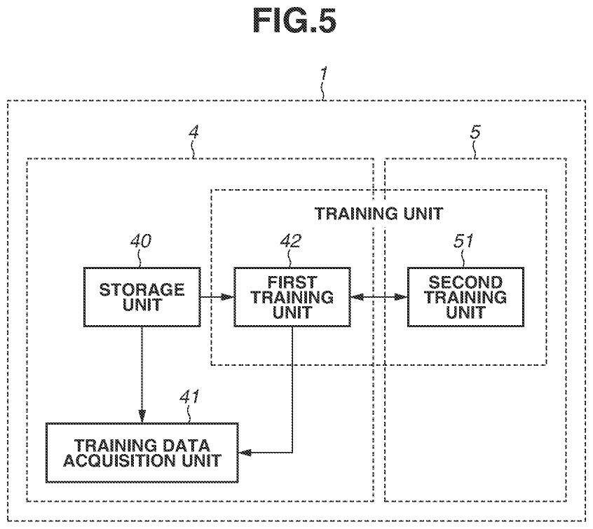

A functional configuration of an information processing system 1 according to the present exemplary embodiment will be described with reference to FIG. 5.

The training processing may be performed before the inference of the inference model in the first exemplary embodiment, or the inference model may be additionally trained through the training processing after the inference.

In the present exemplary embodiment, the information processing system 1 includes a storage unit 40 that stores training data and information about an inference model. The information processing system 1 further includes a training data acquisition unit 41 configured to acquire the training data from the storage unit 40, and a first training unit 42 configured to learn the first partial model on the basis of the acquired training data. Here, a training unit in the information processing system 1 is configured to include the first training unit 42 that performs training processing on the first partial model and a second training unit 51 that performs training processing on the second partial model.

The training unit trains the group of intermediate layers and the first output layer by inputting training data constituting the training data. The training unit trains the group of intermediate layers and the first output layer by backpropagation using loss information calculated by using ground truth data and the first output layer, and trains the inference model by using a parameter relating to the first output layer obtained by the training as a parameter relating to the second output layer.

With such a configuration, the information processing system 1 can perform the training processing of the inference model while securing the confidentiality of the medical data including the training data.

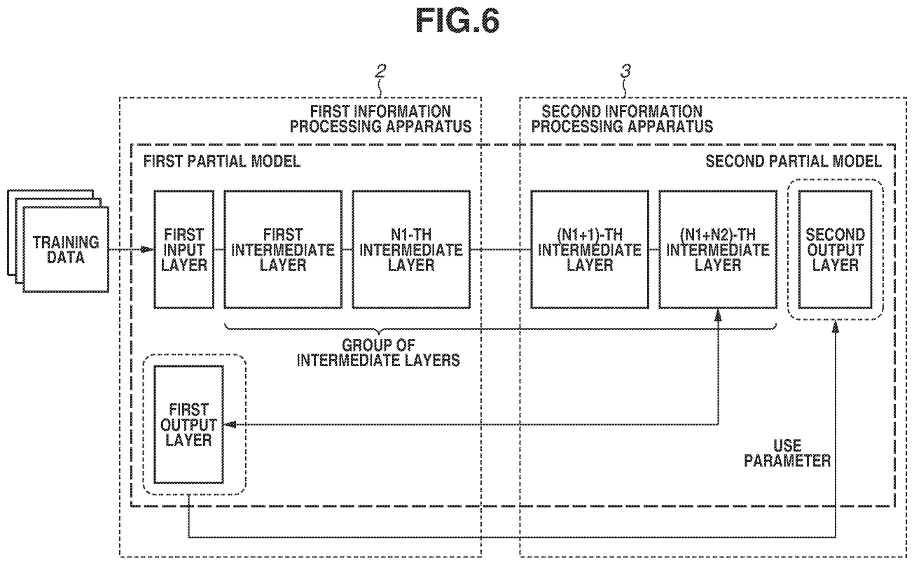

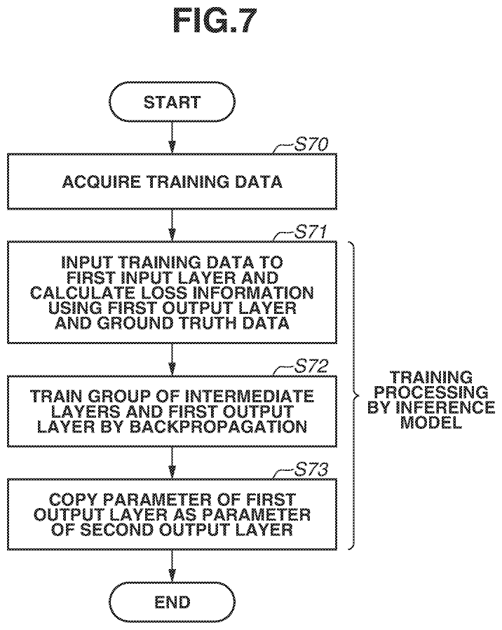

The training processing of the inference model according to the present exemplary embodiment will be described below with reference to FIGS. 6 and 7.

In step S70, the training data acquisition unit 41 acquires, from the storage unit 40, training data in which training data and ground truth data are paired. The training data acquisition unit 41 transmits information about the training data to the first training unit 42, and the processing proceeds to the next step.

In step S71, the first training unit 42 inputs training data to the first input layer and calculates the loss information using an output obtained by forward propagation through the first intermediate layer, the N1-th intermediate layer, the (N1+1)-th intermediate layer, and the (N1+N2)-th intermediate layer, which constitute the group of intermediate layers, and the ground truth, and the processing proceeds to the next step.

In step S72, when the first training unit 42 trains the first output layer and the group of intermediate layers by backpropagation using the calculated loss information, the first training unit 42 transmits a parameter relating to the first output layer to the second training unit 51, and the processing proceeds to the next step. The processing may proceed to the next step after the training processing up to this step is repeated with regard to predetermined training data or the number of epochs, or the processing of the next step may be executed for each epoch or for each predetermined training processing.

In step S73, the second training unit 51 copies the parameter relating to the first output layer and uses the parameter as a parameter relating to the second output layer to determine the parameter relating to the second output layer, and then ends the training process. The parameter relating to the second output layer may not be a copy of the parameter relating to the first output layer, but may be determined by the second information processing apparatus 3 using the loss information by the first training unit 42.

With the above configuration, it is not necessary to transmit both the ground truth data and the training data constituting the training data from the apparatus of the user of the inference model to the outside, and thus it is possible to secure the confidentiality of the medical data including the training data. Further, since a part of the inference model is concealed in the second information processing apparatus 3, which is the information processing apparatus of the provider of the inference model, it is possible to secure the confidentiality of the inference model.

In the third exemplary embodiment, inference processing using an inference model having a network configuration different from that of the first exemplary embodiment will be described. A description of a configuration similar to the configuration in the first exemplary embodiment will be omitted as appropriate. As for a functional configuration, in addition to the configuration illustrated in FIG. 1, the second information processing apparatus 3 may further include an inference target data acquisition unit for verification.

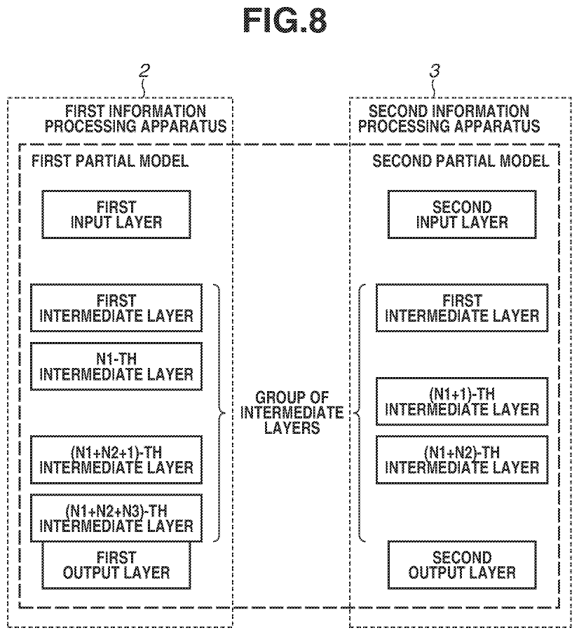

An inference model according to the present exemplary embodiment will be described with reference to FIG. 8. The inference model according to the present exemplary embodiment is an inference model based on a neural network including a first input layer, a second input layer, a group of intermediate layers including a first intermediate layer, an N1-th intermediate layer, an (N1+1)-th intermediate layer, an (N1+N2)-th intermediate layer, an (N1+N2+1)-th intermediate layer, and an (N1+N2+N3)-th intermediate layer, a first output layer, and a second output layer. Then, in an information processing system 1, a first partial model having a part of the configuration of the inference model is arranged in the first information processing apparatus 2, and a second partial model having a part of the configuration of the inference model is arranged in the second information processing apparatus 3. The first partial model includes the first input layer, a first group of intermediate layers including at least one of the intermediate layers in the group of intermediate layers, and the first output layer. For example, as illustrated in FIG. 8, the first group of intermediate layers includes N1 intermediate layers from the first intermediate layer to the N1-th intermediate layer, and N3 intermediate layers from the (N1+N2+1)-th intermediate layer to the (N1+N2+N3)-th intermediate layer. The second partial model includes the second input layer, a second group of intermediate layers including intermediate layers different from those in the first group of intermediate layers in the group of intermediate layers, and the second output layer. For example, as the second group of intermediate layers, as illustrated in FIG. 8, there are N1+N2 intermediate layers from the first intermediate layer to the (N1+N2)-th intermediate layer.

In this case, the (N1+1)-th intermediate layer to the (N1+N2)-th intermediate layer constitute the group of intermediate layers different from the first group of intermediate layers. When the user performs inference processing using the inference model, the user performs the inference processing using the first input layer, the group of intermediate layers, and the first output layer provided in the first information processing apparatus 2. On the other hand, when the provider of the inference model executes the inference processing using the inference target data for verification, the provider executes the inference processing using the second input layer, the group of intermediate layers, and the second output layer.

The intermediate layers constituting the group of intermediate layers are not limited to this pattern.

For example, the first partial model may not include N3 intermediate layers from (N1+N2+1)-th intermediate layer to (N1+N2+N3)-th intermediate layer. Further, the second partial model in the second information processing apparatus 3 of the server may have first, N1-th, (N1+1)-th, (N1+N2+1)-th, and (N1+N2+N3)-th intermediate layers which are the group of intermediate layers constituting the neural network, and the first partial model of the user of the inference model may be a partial model including some of the intermediate layers of the group of intermediate layers. With this configuration, verification of the inference model of the server can be performed by the information processing apparatus of the server.

While the number of intermediate layers constituting the group of intermediate layers or the configurations of the information processing apparatuses can be appropriately changed, since the first to N1-th intermediate layers are provided in the first partial model constituting the first information processing apparatus 2, which is the information processing apparatus of the user of the inference model, the inference and training processing can be performed without transmitting the medical data to the information processing apparatus of the provider of the inference model.

The number of intermediate layers constituting the group of intermediate layers may be any number, and data transmission between the information processing apparatuses may be performed a plurality of times.

With such a configuration, the information processing system 1 can further secure the confidentiality of the inference target data for verification by the provider of the inference model in addition to securing the confidentiality of the inference target data and the inference model.

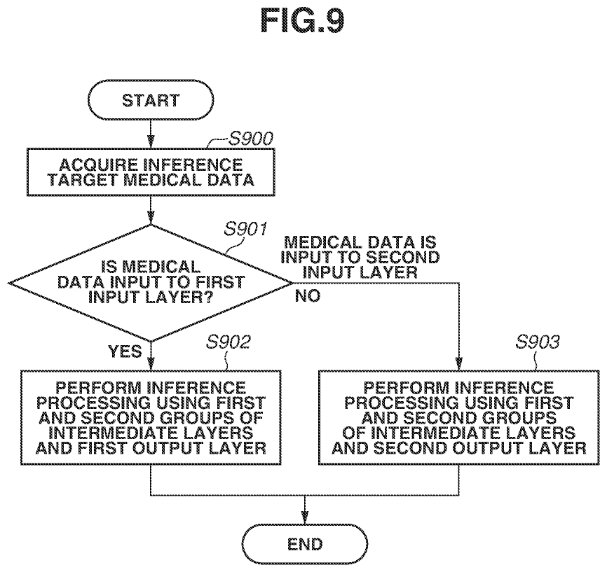

Hereinafter, an inference procedure performed by the information processing system 1 according to the present exemplary embodiment will be described with reference to FIG. 9.

In step S900, the inference target data acquisition unit 21 acquires the inference target medical data from the storage unit 20, and the processing proceeds to the next step.

In step S901, the inference unit determines a path depending on whether the acquisition or input of the inference target medical data is performed in the first input layer of the first partial model constituting the first information processing apparatus 2 or in the second input layer of the second partial model constituting the second information processing apparatus 3. Specifically, when the inference target medical data is input to the first input layer, inference processing using a first path, which performs the inference processing using the group of intermediate layers and the first output layer, is performed.

In the example of the FIG. 8, the group of intermediate layers constituting the first path is intermediate layers from the first intermediate layer to the N1-th intermediate layer of the first partial model, from the (N1+1)-th intermediate layer to the (N1+N2)-th intermediate layer of the second partial model, and from the (N1+N2+1)-th intermediate layer to the (N1+N2+N3)-th intermediate layer of the first partial model. When the inference target medical data for verification is input to the second input layer, inference processing using a second path, which performs the inference processing using the group of intermediate layers and the second output layer, is performed. In the example of FIG. 8, the group of intermediate layers constituting the second path includes intermediate layers from the first intermediate layer to the (N1+N2)-th intermediate layer of the second partial model, and from the (N1+N2+1)-th intermediate layer to the (N1+N2+N3)-th intermediate layer of the first partial model. In other words, the inference unit advances the processing to step S902 when the inference target medical data is input to the first input layer (YES in step S901), and advances the processing to step S903 when the inference target medical data is input to the second input layer (NO in step S901).

In step S902, the inference unit performs the inference processing using the first path by using the first group of intermediate layers and the second group of intermediate layers, which are the groups of intermediate layers constituting the inference model, and the first output layer. With this configuration, the user of the inference model does not have to transmit the inference target medical data and the inference result to the second information processing apparatus 3, which is the information processing apparatus of the provider of the inference model, and the provider of the inference model can secure the confidentiality of the inference model by concealing some of the intermediate layers. After performing the inference processing, the information processing system 1 ends the procedure.

In step S903, the inference unit performs inference processing using the second path by using the first group of intermediate layers and the second group of intermediate layers, which are the groups of intermediate layers constituting the inference model, and the second output layer. With this configuration, in addition to securing the confidentiality of the inference model by concealing some of the intermediate layers of the inference model, even when the inference model is updated by the training processing according to the second exemplary embodiment or the fourth exemplary embodiment to be described below, the provider of the inference model can make an inference without transmitting the provider's own inference target data for verification to the first information processing apparatus 2, which is the information processing apparatus of the user of the inference model. With this configuration, an effect of preventing an unintended decrease in accuracy of the inference model is also exerted.

In the above-described exemplary embodiments, the configuration in which the inference unit outputs an inference result using one of the first and second output layers has been described.

In a first modification, a configuration in which the inference unit outputs an inference result using both the first and second output layers will be described. Assume that the inference processing according to the third exemplary embodiment is executed with regard to the inference target data for verification. The path of the inference processing is not limited to this example.

In the present modification, the tasks of the first output layer and the second output layer may be different. Specifically, the first output layer constituting the first partial model in the first information processing apparatus 2, which is the information processing apparatus of the user, performs segmentation processing on the image data, and the second output layer constituting the second partial model in the second information processing apparatus 3, which is the information processing apparatus of the provider of the inference model, performs classification processing on the image data. With this configuration, the provider of the inference model can detect unintended use of the inference model by a plurality of times of inference processing, and the user of the inference model can acquire an inference result of a highly confidential task, such as segmentation processing, only by the first information processing apparatus 2. Therefore, the confidentiality of the medical data can be secured. As the inference model used in such a case, a multi-task inference model, such as Mask Region-based Convolutional Neural Networks (Mask R-CNN), of a known technique can be applied. In the multi-task inference model, some tasks having high confidentiality are provided in the information processing apparatus of the user of the inference model, and some tasks are provided in the information processing apparatus of the provider of the inference model, whereby in addition to the confidentiality of the inference model and the inference target data, inference processing by an unintended inference model can be prevented.

In the fourth exemplary embodiment, training processing using an inference model having a network configuration different from that of the second exemplary embodiment will be described. A description of a configuration similar to the configuration in the second exemplary embodiment will be omitted as appropriate. As for a functional configuration, in addition to the configuration illustrated in FIG. 5, the second information processing apparatus 3 may include a training data acquisition unit or an inference target data acquisition unit for verification.

An inference model according to the present exemplary embodiment will be described with reference to FIG. 8. Similar to the third exemplary embodiment, the inference model according to the present exemplary embodiment is an inference model based on a neural network including a first input layer, a second input layer, a group of intermediate layers including an N-th intermediate layer, an (N+1)-th intermediate layer, an (N+2)-th intermediate layer, and an (N+3)-th intermediate layer, a first output layer, and a second output layer.

In the information processing system 1, a first partial model having a part of the configuration of the inference model is arranged in the first information processing apparatus 2, and a second partial model having a part of the configuration of the inference model is arranged in the second information processing apparatus 3. The first partial model includes the first input layer, the second input layer, a first group of intermediate layers including at least one of the intermediate layers in the group of intermediate layers, and the first output layer. The second partial model includes the second input layer, a second group of intermediate layers including intermediate layers different from those in the first group of intermediate layers in the group of intermediate layers, and the second output layer.

When the user trains the inference model, the first input layer, the group of intermediate layers, and the first output layer provided in the first information processing apparatus 2, which is the information processing apparatus of the user, of the inference model are used to perform the training processing. The training processing is completed by using a parameter relating to the first output layer determined by the training processing and a parameter relating to the first intermediate layer in the first information processing apparatus 2 as parameters for the second output layer and the first intermediate layer in the second information processing apparatus 3.

On the other hand, when the provider of the inference model trains the inference model, the second input layer, the group of intermediate layers, and the second output layer provided in the second information processing apparatus 3, which is the information processing apparatus of the provider of the inference model, of the inference model are used to perform the training processing. The training processing is completed by using a parameter relating to the second output layer determined by the training processing and a parameter relating to the first intermediate layer in the second information processing apparatus 3 as parameters for the first output layer and the first intermediate layer in the first information processing apparatus 2.

The information processing system 1 is configured as described above, and thus can secure, in addition to the confidentiality of the inference model, the confidentiality of medical data since it is not necessary to transmit any of the ground truth data and the training data constituting the training data to an external device. In addition, the provider of the inference model can make an inference on the inference target data for verification of the model by using the second input layer, the group of intermediate layers, and the second output layer, and thus it is possible to verify whether the training processing by the user of the inference model or the training processing by the provider of the inference model is appropriately performed.

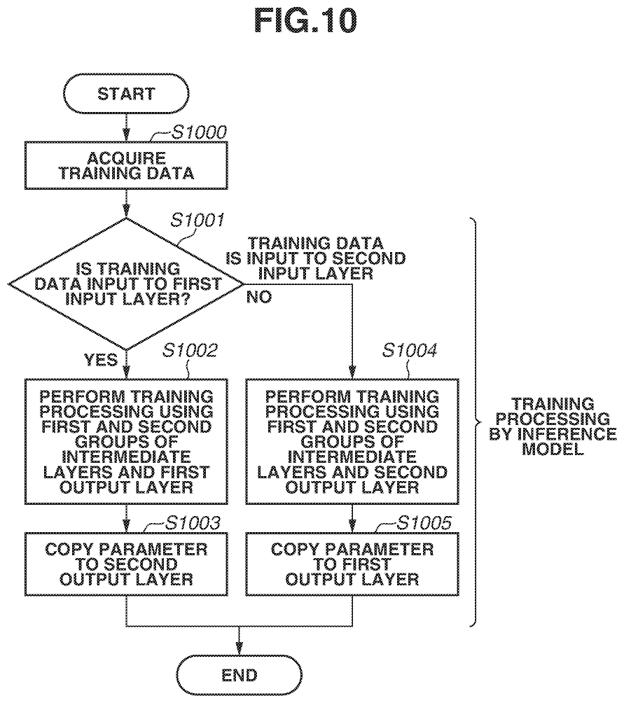

Hereinafter, a training procedure performed by the information processing system 1 according to the present exemplary embodiment will be described with reference to FIG. 10.

In step S1000, the training data acquisition unit 41 acquires training data from the storage unit 40, and the processing proceeds to the next step.

In step S1001, the training unit determines a path for executing the inference processing in the inference model depending on whether the acquisition of the training data or input of the training data constituting the training data is an input to the first input layer of the first partial model constituting the first information processing apparatus 2 or an input to the second input layer of the second partial model constituting the second information processing apparatus 3. Specifically, when training data is input to the first input layer, the training processing using the first path that performs the inference processing using the group of intermediate layers and the first output layer is performed, and when the training data is input to the second input layer, the training processing using the second path that performs the inference processing using the group of intermediate layers and the second output layer is performed. In other words, the training unit advances the processing to step S1002 when the training data is input to the first input layer (YES in step S1001), and advances the processing to step S1004 when the training data is input to the second input layer (NO in step S1001).

In step S1002, the training unit performs forward propagation of the training data to the first input layer, the first group of intermediate layers, the second group of intermediate layers, and the first output layer. In addition, the training unit calculates loss information between an output from the first output layer and ground truth data, and a parameter relating to each of the layers is determined by backpropagation based on the loss information.

In step S1003, the parameter relating to the first output layer is copied and used as a parameter relating to the second output layer, and the processing is terminated.

In step S1004, the training unit performs forward propagation of the training data to the second input layer, the first group of intermediate layers, the second group of intermediate layers, and the second output layer. In addition, the training unit calculates loss information between an output from the second output layer and ground truth data, and a parameter relating to each of the layers is determined by backpropagation based on the loss information.

In step S1005, the parameter relating to the second output layer is copied and used as a parameter relating to the first output layer, and the processing is terminated. The parameter relating to the first output layer or the second output layer may not be a copy of the parameter relating to one output layer, and may be determined by training in another information processing apparatus using the loss information calculated in one information processing apparatus.

The information processing system 1 is configured as described above, and thus can secure, in addition to the confidentiality of the inference model, the confidentiality of medical data since it is not necessary to transmit any of the ground truth data and the training data constituting the training data to an external device. In addition, the provider of the inference model can make an inference on the inference target data for verification of the model by using the second input layer, the group of intermediate layers, and the second output layer, and thus it is possible to verify whether the training processing by the user of the inference model or the training processing by the provider of the inference model is appropriately performed.

In the training processing according to the second and fourth exemplary embodiments, the case where the training processing of the inference model is performed using backpropagation has been described.

In a second modification, a case where the training processing is performed by a training method other than backpropagation in the training processing of the inference model will be described.

For example, the method may be a method that uses synthetic gradients to train a model for estimating a gradient to be obtained for each layer, a method that uses feedback alignment in which a fixed random matrix is used in backpropagation, a method that uses target propagation that propagates targets instead of errors, or any other method.

The present disclosure can also be realized by executing the following processing. Specifically, the processing is processing in which software (a program) for implementing the functions of the above described exemplary embodiments is supplied to a system or an apparatus via a network or various types of storage medium, and a computer (or a CPU, or a micro processing unit (MPU)) of the system or the apparatus reads and executes the program.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2022-128577, filed Aug. 12, 2022, which is hereby incorporated by reference herein in its entirety.

What is claimed is:

1. An information processing system comprising:
a first information processing apparatus;
a second information processing apparatus configured to communicate with the first information processing apparatus via a network;
an inference target data acquisition unit configured to acquire inference target data; and
an inference unit configured to perform predetermined inference processing on the inference target data by using a first partial model and a second partial model,
wherein the information processing system performs predetermined inference processing by using an inference model based on a neural network including a first input layer, a group of intermediate layers, a first output layer, and a second output layer, the first output layer and the second output layer being provided in different information processing apparatuses,
wherein the first information processing apparatus includes the first partial model configured to include the first input layer, a first group of intermediate layers including at least one of the intermediate layers in the group of intermediate layers, and the first output layer, and
wherein the second information processing apparatus includes the second partial model configured to include a second group of intermediate layers including an intermediate layer different from the intermediate layers included in the first group of intermediate layers in the group of intermediate layers, and the second output layer.

2. The information processing system according to claim 1, wherein the inference unit performs the predetermined inference processing on the inference target data input to the first input layer by using the group of intermediate layers, the first output layer, and the second output layer.

3. The information processing system according to claim 1,
wherein the neural network further includes a second input layer, and
wherein the second partial model further includes the second input layer.

4. The information processing system according to claim 3,
wherein, in a case where the inference target data is input to the first input layer, the inference unit performs the predetermined inference processing on the inference target data by using at least the first input layer, the first group of intermediate layers, the second group of intermediate layers, and the first output layer, and
wherein, in a case where the inference target data is input to the second input layer, the inference unit performs the predetermined inference processing on the inference target data by using at least the second input layer, the first group of intermediate layers, the second group of intermediate layers, and the second output layer.

5. An information processing system comprising:
a first information processing apparatus;
a second information processing apparatus configured to communicate with the first information processing apparatus via a network;
a training data acquisition unit configured to acquire training data; and
a training unit configured to learn a first partial model and a second partial model by using the training data,
wherein the information processing system performs training processing of training an inference model based on a neural network including a first input layer, a group of intermediate layers, a first output layer, and a second output layer, the first output layer and the second output layer being provided in different information processing apparatuses,
wherein the first information processing apparatus includes the first partial model configured to include the first input layer, a first group of intermediate layers including at least one of the intermediate layers in the group of intermediate layers, and the first output layer, and
wherein the second information processing apparatus includes the second partial model configured to include a second group of intermediate layers including an intermediate layer different from the intermediate layers included in the first group of intermediate layers in the group of intermediate layers, and the second output layer.

6. The information processing system according to claim 5,
wherein the training unit inputs training data constituting the training data to the first input layer of the inference model, and trains the group of intermediate layers and the first output layer, and
wherein the training unit trains the inference model by training the group of intermediate layers and the first output layer by backpropagation using loss information calculated using ground truth data constituting the training data and the first output layer, and by using a parameter relating to the first output layer obtained by the training as a parameter relating to the second output layer.

7. The information processing system according to claim 5,
wherein the neural network further includes a second input layer, and
wherein the second partial model further includes the second input layer.

8. The information processing system according to claim 7,
wherein, in a case where training data constituting the training data is input to the first input layer, the training unit trains the inference model by training the group of intermediate layers and the first output layer by backpropagation using loss information calculated using ground truth data constituting the training data and the first output layer, and by using a parameter relating to the first output layer obtained by the training as a parameter relating to the second output layer, and
wherein, in a case where training data constituting the training data is input to the second input layer, the training unit trains the inference model by training the group of intermediate layers and the second output layer by backpropagation using loss information calculated using ground truth data constituting the training data and the second output layer, and by using a parameter relating to the second output layer obtained by the training as a parameter relating to the first output layer.

9. An information processing system comprising:
a first information processing apparatus; and
a second information processing apparatus configured to communicate with the first information processing apparatus via a network,
wherein the information processing system performs at least one of inference processing and training processing by using an inference model based on a neural network including a first input layer, a second input layer, a group of intermediate layers, a first output layer, and a second output layer, the first input layer and the second input layer being provided in different information processing apparatuses, and
wherein the information processing system is configured to perform at least one of the inference processing and the training processing on the inference model using a path corresponding to the input layer to which target data is input.

10. The information processing system according to claim 9, wherein the path includes a first path using the first input layer, the group of intermediate layers, and the first output layer, and a second path using the second input layer, the group of intermediate layers, and the second output layer.

11. The information processing system according to claim 9, wherein the first output layer and the second output layer are provided in different information processing apparatuses.

12. The information processing system according to claim 11,
wherein the first input layer and the first output layer are provided in the same information processing apparatus, and
wherein the second input layer and the second output layer are provided in the same information processing apparatus.
